(19) United States
(12) Patent Application Publication
(10) Pub. No.: US 2009/0281418 A1
(43) Pub. Date: Nov. 12, 2009

(54) DETERMINING TISSUE SURROUNDING AN OBJECT BEING INSERTED INTO A PATIENT

(75) Inventors: Daniel Simon Anna Ruijters, Eindhoven (NL); Drazenko Babic, Best (NL); Robert Johannes Frederik Homan, Batenburg (NL); Pieter Maria Mielenkamp, Veldhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTOMICS N.V., Eindhoven (NL)

(21) Appl. No.: 12/295,754

(22) PCT Filed: Mar. 15, 2007
(86) PCT No.: PCT/IB07/50897

§ 371 Date: Oct. 2, 2008

(30) Foreign Application Priority Data
Apr. 3, 2006 (EP) 06112145.5

(51) Int. Cl. A61B 5/05; G06K 9/00

(52) U.S. Cl. 600/424; 382/131; 382/130; 382/154; 382/294

(57) ABSTRACT

It is described a method for determining and assessing the tissue surrounding an object being inserted into a patient. The method comprises acquiring a first dataset representing a first 3D image of the patient, acquiring a second dataset representing a second 3D image of the blood vessel structure of the patient and acquiring a third dataset representing a 2D image of the patient including the object. The method further comprises recognizing the object within the 2D image, registering two of the three datasets with each other, whereby the object is back-projected in the blood vessel structure, in order to generate a first combined dataset, and registering the first combined dataset with the remaining dataset in order to generate a second combined dataset representing a further image surrounding the object. The method allows for combining diagnostic scanning such as CT, 3D RA and real-time 2D fluoroscopy. Thereby, it is possible to generate an image perpendicular to a catheter tip representing the object being inserted into the patient. Since the 3D-RA displays the lumen and the diagnostic scanning displays soft-tissue, it is possible to assess the tissue at the catheter tip position.

The present invention relates to the field of digital image processing, in particular digital image processing for medical purposes, wherein datasets obtained with different examination methods are registered with each other.

Specifically, the present invention relates to a method for determining and assessing the tissue surrounding an object being inserted into a patient.

Further, the present invention relates to a data processing device for determining and assessing the tissue surrounding an object being inserted into a patient.

Furthermore, the present invention relates to a computer-readable medium and to a program element having instructions for executing the above-mentioned method for determining and assessing the tissue surrounding an object being inserted into a patient.

In many technical applications, the problem occurs of making an object visible that has penetrated into a subject with respect to its position and orientation within the subject. In medical technology there is, for example, a problem of this sort in the treatment of tissue from inside the body of a living being, using a catheter which is to be guided by a physician to the point of the tissue to be examined in a manner that is as precise and closely monitored as possible. As a rule, guidance of the catheter is accomplished using an imaging system, for example a C-arm X-ray apparatus, or an ultrasound apparatus, with which images can be obtained of the interior of the body of the living subject, wherein these images indicate the position and orientation of the catheter relative to the tissue to be examined.

An advantage of the use of an X-ray CT apparatus as an imaging system in the catheter procedure is that good presentation of soft tissue parts occurs in images obtained using an X-ray CT apparatus. In this way, the current position of the catheter relative to the tissue to be examined can be visualized and measured.

U.S. Pat. No. 6,546,279 B1 discloses a computer controlled system for guiding a needle device, such as a biopsy needle, by reference to a single mode medical imaging system employing any one of CT imaging equipment, magnetic resonance imaging equipment, fluoroscopic imaging equipment, or three-dimensional (3D) ultrasound system, or alternatively, by reference to a multi-modal imaging system, which includes any combination of the aforementioned systems. The 3D ultrasound system includes a combination of an ultrasound probe and both passive and active infrared tracking systems so that the combined system enables a real time image display of the entire region of interest without probe movement.

U.S. Pat. No. 6,317,621 B1 discloses a method and an apparatus for catheter navigation in 3D vascular tree exposures, in particularly for inter-cranial application. The catheter position is detected and mixed into the 3D image of the pre-operatively scanned vascular tree reconstructed in a navigation computer and an imaging (registering) of the 3D patient coordination system ensues on the 3D image coordination system prior to the intervention using a number of markers placed on the patient's body, the position of these markers being registered by the catheter. The markers are detected in at least two two-dimensional (2D) projection images, produced by a C-arm X-ray device, from which the 3D angiogram is calculated. The markers are projected back on to the imaged subject in the navigation computer and are brought into relation to the marker coordinates in the patient coordinate system, using projection matrices applied to the respective 2D projection images, wherein these matrices already have been determined for the reconstruction of the 3D volume set of the vascular tree.

US 2001/0029334 A1 discloses a method for visualizing the position and the orientation of an object that is penetrating, or that has penetrated, into a subject. Thereby, a first set of image data are produced from the interior of the subject before the object has penetrated into the subject. A second set of image data are produced from the interior of the subject during or after the penetration of the object into the subject. Then, the sets of image data are connected and are superimposed to form a fused set of image data. An image obtained from the fused set of image data is displayed.

The described visualizing method allows to obtain the 3D position and the orientation of the object inserted in the patient out of two 2D X-ray projections which are both registered to a dataset acquired by means of CT. This has the disadvantage that when carrying out the described visualizing method (a) the inserted object must not be moved and (b) X-ray equipment has to be moved around the patient in order to make two 2D x-ray recordings obtained at different angles. Thus, the described visualizing method is rather time consuming.

There may be a need for precisely and less time consuming method for determining tissue surrounding an object being inserted into a patient.

This need may be met by the subject matter according to the independent claims. Advantageous embodiments of the present invention are described by the dependent claims.

According to a first aspect of the present invention there is provided a method for determining the tissue surrounding an object being inserted into a patient. The described the method comprises the steps of (a) acquiring a first dataset representing a first three-dimensional (3D) image of the patient, (b) acquiring a second dataset representing a second 3D image of the blood vessel structure of the patient and (c) acquiring a third dataset representing a two-dimensional (2D) image of the patient including the object being inserted into the patient. The described method further comprises the steps of (d) recognizing the object within the 2D image, (e) registering two of the three datasets with each other in order to generate a first combined dataset, and (f) registering the first combined dataset with the remaining dataset in order to generate a second combined dataset representing a further image surrounding the object.

This aspect of the invention is based on the idea that an indirect two-step registration whereby first two dataset are superimposed with each other and later on the remaining dataset is merged with the first combined dataset is much more reliable and much more robust compared to a direct one-step projection of the third dataset onto the first dataset.

Preferably, the second dataset is acquired by means of a second examination method which is from a physical point of view similar to a third examination method yielding the third dataset. This means, that the second examination method and the third examination method both use the same or at least similar spectral electromagnetic radiation such that the physical interaction between this radiation and the patients body is more or less the same for both examination methods.

In this respect the term “registration” means, that the spatial relation between two datasets is established. The term “combined datasets” denotes here the individual datasets and their registration(s).

It has to be noted that from the second combined dataset, there may be extracted 2D or alternatively 3D images showing the patients tissue surrounding the object.

According to an embodiment of the present invention (a) the step of registering two of the three datasets with each other comprises registering the third dataset with the second dataset in order to generate the first combined dataset representing an image surrounding the object, whereby the object is back-projected in a 3D structure, contained in the second dataset, e.g. the blood vessels, and (b) the step of registering the first combined dataset with the remaining dataset comprises registering the first combined dataset with the first dataset.

This has the advantage that the spatial position of the inserted object may define a region of interest surrounding the object. Therefore, further registering procedures may be restricted to regions corresponding to the region of interest. Thus, the required computational effort may be reduced significantly.

However, it has to be pointed out that in particular when the registering is carried out only within a small region of interest, it has to be ensured that the corresponding datasets include enough landmarks.

According to a further embodiment of the present invention (a) the step of registering two of the three datasets with each other comprises registering the first dataset with the second dataset in order to generate the first combined dataset and (b) the step of registering the first combined dataset with the remaining dataset comprises registering the first combined dataset with the third dataset.

This may have the advantage that the first registering procedure is carried out with two datasets both representing a 3D image. Therefore, within the second registering procedure the third dataset representing a 2D image is projected onto the first combined dataset representing detailed information of the patient under study or at of a region of interest within the body of the patient.

It has to be mentioned that it is also possible to generate first two combined datasets and later on to merge these two combined datasets with each other. In this case a first combined dataset may be generated by registering the third dataset with the second dataset and a second combined dataset may be generated by registering the second dataset with the first dataset.

According to a further embodiment of the present invention the object is a catheter being inserted into a vessel of the patient. This may provide the advantage that a catheter tip may be moved within the patients vessel system by means of a minimal invasive medical examination technique. Thereby, many different parts of the patients body may be examined or treated, wherein by means of a minimal invasive technique an appropriate catheter is inserted at only one single insertion point.

According to a further embodiment of the present invention the method further comprises the step of creating a cross-sectional view surrounding the catheter based on the second combined dataset. Preferably, the cross-sectional view is generated at a position corresponding to a tip of the catheter. The 3D position of the catheter tip is determined by back-projecting the catheter tip recognized in the 2D image on the 3D vessel tree structure obtained by the acquisition of the second dataset.

Therefore, the composition of the tissue surrounding the tip of the catheter may be determined. This is in particular beneficial when the front part of the catheter represents a tool for directly carrying out a medical treatment within or in a close surrounding of the corresponding vessel section.

According to a further embodiment of the present invention the cross-sectional view is oriented perpendicular to the tangent of a section of the vessel, in which section the catheter is inserted. This may provide the advantage that an image projection or image slice is selected, which allows for a precise determination of the tissue surrounding the catheter tip with a high spatial resolution and contrast resolution.

Further, this may allow that a cross-section through the catheter tip position, which plane comprises a normal corresponding to the tangent of the catheter tip, can be displayed in real-time. This means, when the catheter is moved along the corresponding vessel, the cross-section moves uniformly along with it and the tissue surrounding the catheter tip can be assessed in real-time.

According to a further embodiment of the present invention the first dataset is obtained by means of computed tomography (CT) and/or by means of magnetic resonance (MR). This has the advantage that the whole patient may be examined by means of well-known medical examination procedures.

According to a further embodiment of the present invention the first dataset is acquired before the object has been inserted into the patient. Thereby, it is possible to determine a 3D representation of the patient in an unperturbed state i.e. without the catheter being inserted.

It has to be mentioned that in particular when the first dataset is acquired by means of CT or MR, one can obtain a pre-interventional data set representing the patient's soft tissue.

According to a further embodiment of the present invention the second dataset is obtained by means of 3D rotational angiography (RA). Thereby, an appropriate contrast agent is used which has to be inserted into the patients vessel system preferably shortly before the rotational angiography is carried out.

According to a further embodiment of the present invention the second dataset is obtained by means of computed tomography angiography (CTA) and/or by means of magnetic resonance angiography (MRA). The CTA respectively the MRA datasets can directly be registered with a 2D x-ray dataset using image-based registration. Thereby, the object can be back-projected on the vessel tree structure, which has been segmented from the CTA or MRA.

At this point it has to be mentioned that in case a CTA and/or a MRA is used for acquiring the second dataset also the soft-tissue of the patient is already visible in the CTA/MRA images. Therefore, the second dataset comprises both the information of the first dataset and the second dataset. This means that the second dataset can be interpreted as an already combined dataset such that the use of the individual first dataset is optional.

According to a further embodiment of the present invention the second dataset is limited to a region of interest surrounding the object. This has the advantage that only a relevant portion of the patient's blood vessel structure may be included in the second 3D image such that the computationally effort can be limited without having a negative impact on the quality of the further image.

According to a further embodiment of the present invention the second dataset also comprises segmented images of the patient's blood vessel structure. The segmented blood vessel structure, combined with the a-priori knowledge that the object is contained within this structure, allows the determination of the 3D position of the object from the combination of the second dataset and the third dataset.

According to a further embodiment of the present invention the first combined dataset represents a 3D image. This has the advantage that the position of the object being identified within the 2D image may be combined with the second dataset in such a manner that the position of the object is specified precisely within a 3D image.

Preferably, one has to take into account the a priori knowledge that the object is always positioned within a defined morphological structure, e.g. the blood vessels. Thereby, the position of the object may be rendered within the first combined dataset, which preferably represents a 3D rotational angiography volume being slightly modified by the information originating from the third dataset.

According to a further embodiment of the present invention the third dataset is acquired by means of X-radiation. This has the advantage that a common 2D X-ray imaging method may be applied. Thereby, the 2D X-ray imaging may be carried out with or without contrast agent being inserted into the patient's blood vessel structure. Since a catheter typically is made from a material comprising a strong X-ray attenuation the recognizability of the object is not or only very weakly influenced by the presence of contrast agent.

According to a further embodiment of the present invention the second dataset and the third dataset are acquired by means of the same medical examination apparatus. This has the advantage that the second and the third dataset may be acquired within a short span of time preferably by means of a minimal invasive operation, wherein a catheter is inserted into the patient's blood vessel structure. This provides the basis for an in particular advantageous feature, namely a real time monitoring or tracking of the catheter.

Acquiring the second and the third dataset by means of the same medical examination apparatus has the further advantage that it is rather easy to register these datasets with each other with purely geometrical calculations. This means that the position of the geometry of the apparatus during acquisition serves to generate a registration of the datasets. Since both datasets were acquired by means of the same apparatus, the relation between the coordinate systems of these datasets is known.

It has to be mentioned that of course the overall resolution may be enhanced if the patient is spatially fixed during the acquisition of the third dataset and the second dataset. Preferably, the patient is fixed with regard to a table. This improves a geometry-based registration between the third dataset and the second data set representing a 3D image of the patient's blood vessel structure.

According to a further embodiment of the present invention the object is moved within the patient's blood vessel structure and third datasets are acquired for different positions of the object. Thereby, each third dataset represents a 2D image of the patient including the object being inserted into the patient. For each position of the object there is carried out a data evaluation, which data evaluation comprises (a) recognizing the object within the 2D image and (b) registering the third dataset with the second dataset in order to generate a first combined dataset representing an image surrounding the object, whereby the object is back-projected into the blood vessel structure.

It has to be mentioned that it is not necessary but however possible to supplement the described method by a further step, wherein the data evaluation for each position of the object further comprises registering the first combined dataset with the first dataset in order to generate a second combined dataset representing a further image surrounding the object. This step is optional because when the object is moved within the patient's blood vessel structure both the first and the second dataset do not change.

This has the advantage that the tissue surrounding a moving catheter may be imaged by means of subsequent measuring and data evaluation procedures. In other words, the moving catheter and its surrounding tissue may be monitored in real time and it is possible to perform the described method on a stream comprising a series of 2D X-ray images. Then the position of the catheter tip in the 3D vessel tree can be localized more robustly, since we know that the catheter does not suddenly jump from one vessel to another.

It has to be pointed out that it is not necessary to obtain multiple 3D RA datasets representing the second datasets. Preferably, a lot of 2D x-ray images or stream of 2D x-ray images is mapped on one single 3D RA dataset.

Therefore, only one 3D RA data acquisition is necessary. This has the advantage that an extra amount of contrast medium and x-ray dose both being harmful for the patient may be avoided.

By combining (a) a 3D catheter tracking based on the repeatedly acquired third datasets with (b) the second dataset, the position of the catheter within the 3D vessel structure can be identified permanently. By applying the thereby created first combined dataset with pre-interventional acquired soft tissue data sets representing the first 3D image of the patient, the catheter tip location may be real time linked to the soft tissue cross section, which will allow for real time integration of the vessels visualization and the soft tissue surrounding. This can result in a full understanding of the catheter position within the angiographic data sets with a required link to the surrounding soft tissue.

Preferably, the linking of the 3D catheter position to the surrounding soft tissue information, originating from different soft-tissue modalities, may be used in the following applications:
Determination of the optimal position for intra-arterial particle injection in endovascular embolization of various neoplastic tissues, arteriovenous malformations, etc.
Determination of the optimal position for intra-cranial stents in cases where aneurysms are pressing on surrounding eloquent and motoric brain tissue.
Determination of the vessel portions to be embolized in e.g. a hemorrhagic stroke.

By applying the described method a thrombus location may be visualized, which location is normally not visible in a combined 2D/3D dataset, wherein the combined 2D/3D dataset is based solely on acquired angiographic data. In particular, if a therapeutic treatment is defined and the treatment is going to be performed via a minimal invasive intra-arterial approach, a precise knowledge of the position of the catheter becomes very important. Therefore, merging the 2D/3D X-ray angiographic dataset (i.e. the first combined data set) with the corresponding image of the first 3D image (e.g. obtained by CT) may precisely reveal the location and the extend of the thrombus obstruction.

According to a further aspect of the present invention there is provided a data processing device for determining the tissue surrounding an object being inserted into a patient. The data processing device comprises (a) a data processor, which is adapted for performing the method as set forth in claim 1, and (b) a memory for storing the acquired first dataset, the acquired second dataset, the acquired third dataset and the registered first combined dataset.

According to a further aspect of the invention there is provided a computer-readable medium on which there is stored a computer program for determining the tissue surrounding an object being inserted into a patient. The computer program, when being executed by a data processor, is adapted performing exemplary embodiments of the above-described method.

According to a further aspect of the invention there is provided a program element for determining the tissue surrounding an object being inserted into a patient. The program element, when being executed by a data processor, is adapted for performing exemplary embodiments of the above-described method.

The program element may be written in any suitable programming language, such as, for example, C++ and may be stored on a computer-readable medium, such as a CD-ROM. Also, the computer program may be available from a network, such as the World Wide Web, from which it may be downloaded into image processing units or processors, or any suitable computer.

It has to be noted that embodiments of the invention have been described with reference to different subject matters. In particular, some embodiments have been described with reference to method type claims whereas other embodiments have been described with reference to apparatus type claims. However, a person skilled in the art will gather from the above and the following description that, unless other notified, in addition to any combination of features belonging to one type of subject matter also any combination between features relating to different subject matters, in particular between features of the method type claims and features of the apparatus type claims is considered to be disclosed with this application.

The aspects defined above and further aspects of the present invention are apparent from an example of embodiment to be described hereinafter and are explained with reference to the example of embodiment. The invention will be described in more detail hereinafter with reference to example of embodiment but to which the invention is not limited.

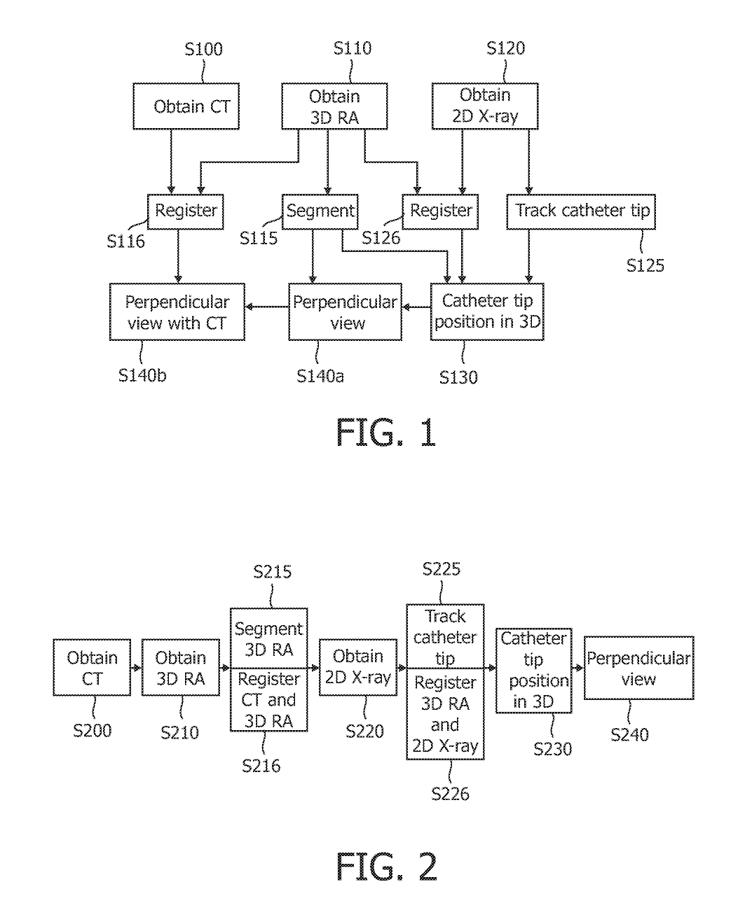

FIG. 1 shows a diagram illustrating different data acquisition and data processing steps according to a preferred embodiment of the invention.

FIG. 2 shows a temporal workflow for carrying out the preferred embodiment of the invention.

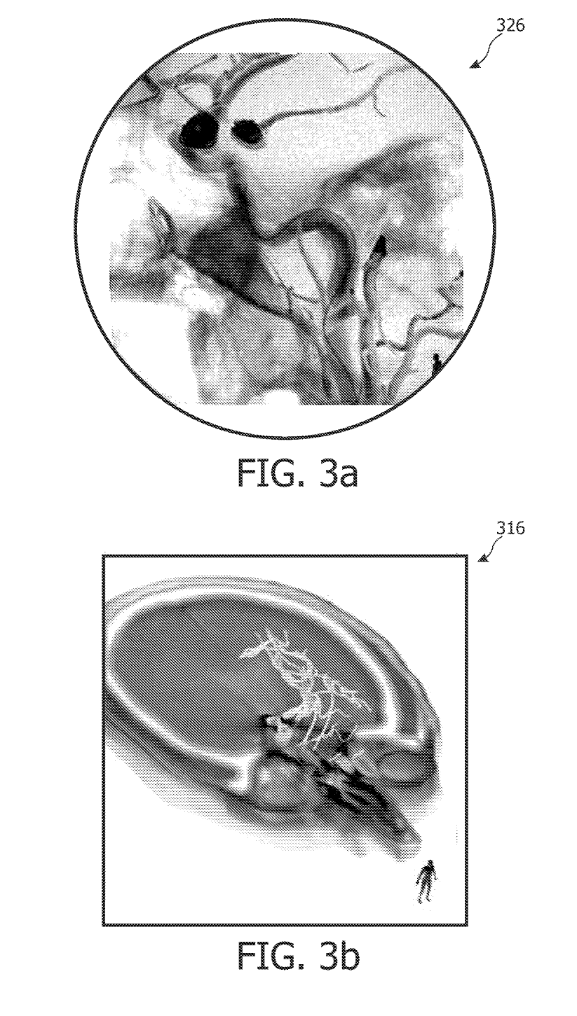

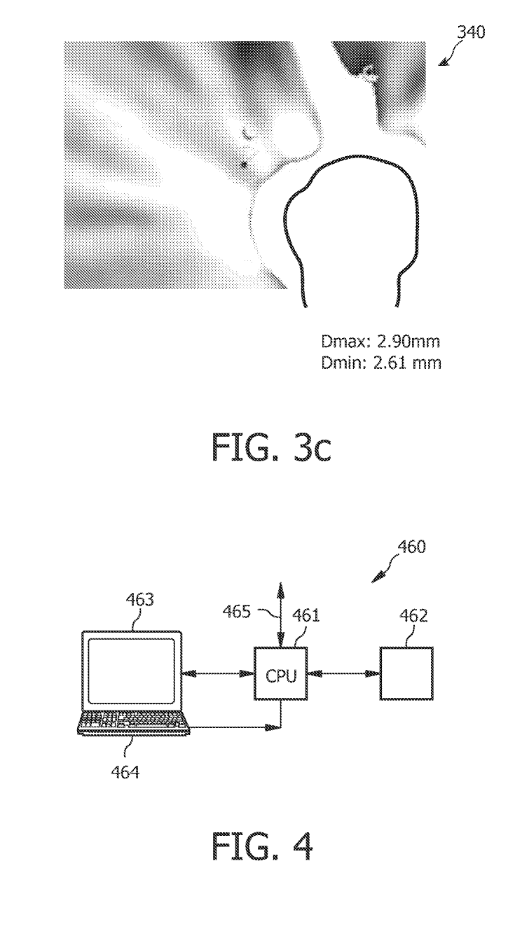

FIGS. 3a, 3b, and 3c show images, which are generated in the course of performing the preferred embodiment of the invention.

FIG. 4 shows an image processing device for executing the preferred embodiment of the invention.

The illustration in the drawing is schematically. It is noted that in different drawings, similar or identical elements or steps are provided with the same reference signs or with reference signs, which are different from the corresponding reference signs only within the first digit.

FIG. 1 shows a diagram illustrating different data acquisition and data processing steps according to a preferred embodiment of the invention. The steps may be accomplished by means of dedicated hardware and/or by means of appropriate software.

In order to determine both precisely and within a short time the tissue surrounding a catheter being inserted into a patient's blood vessel, three different data acquisitions have to be performed.

First, as indicated with a step S100, a patient under examination is subjected to a computed tomography (CT) procedure. Thereby, a CT dataset representing a 3D image of the patient or at least of a region of interest of the patient's body is acquired. Preferably, this procedure is carried out before the catheter is inserted into the patient's vessel structure.

It has to be mentioned that the described method may also be carried out with other 3D diagnostic scanning methods such as e.g. magnetic resonance, positron emission tomography, single photon emission tomography, 3D ultrasound, etc.

Second, as indicated with a step S110, the patient is subjected to a so-called 3D rotational angiography (RA). The 3D RA yields a 3D representation of the patient's blood vessel structure. In order to provide for a precise image an appropriate contrast agent is used. This agent has to be injected in due time before the 3D RA examination is carried out.

Preferably, the 3D RA examination may be realized by employing a well-known C-arm, whereby an X-ray source and an opposing X-ray detector mounted at the C-arm are commonly moved around the patient's body.

Third, as indicated with a step S120, a 2D X-ray image of the patient is recorded. Thereby, the 2D X-ray image may be obtained by common known X-ray fluoroscopy. Preferably, the 2D X-ray recording is carried out by employing the above-mentioned C-arm. The field of view of the 2D X-ray image is adjusted such that the inserted catheter is included within the 2D image. Thereby, as indicated with a step S125, the catheter and in particular the tip of the catheter can be tracked by processing the corresponding 2D X-ray dataset. Since the 2D X-ray recording does not require a rotational movement of the C-arm, the positing of the catheter may be identified very quickly. Therefore, also a moving catheter may be tracked in real time.

At this point it is mentioned that tracking the catheter tip may also be carried out by means of so-called sensor-based tracking of the catheter tip. Thereby, a sophisticated catheter has to be used which is provided with a sender element. This sender element is adapted to send a position finding signal, which can be detected by an appropriate receiver.

Following the above-mentioned data acquisition steps S100, S110 and S120 there are carried out three data processing steps S116, S115 and S126.

First, as indicated with step S116, the dataset generated by means of the CT procedure (step S100) is registered with the dataset generated by means of the 3D RA procedure (step S110). Thereby, the information being included in the CT dataset is spatially combined with the information being included in the 3D RA dataset. In the embodiment described here, the CT information regarding the soft tissue surrounding the patient's vessel structure and the 3D RA information regarding the spatial position of the patient's vessels are of particularly importance.

Second, as indicated with step S115, the 3D RA dataset obtained with step S110 is segmented such that for further processing only the corresponding segments may be used. This reduces the computationally effort of the described method significantly.

Third, as indicated with step S126, the dataset generated by means of the 3D RA procedure (step S110) is registered with the dataset obtained with the 2D X-ray imaging (step S120). Thereby, the information regarding in particular the present position of the catheter being included in the 2D X-ray dataset is combined with the information regarding the 3D vessel structure being included in the 3D RA dataset. In other words, the catheter tip is back-projected on the vessel tree structure obtained by means of 3D RA. This is a very essential step since without this step S126 the 3D location of the catheter tip is unknown and a later on generation of cross sectional views of the catheter tip and the surrounding tissue would not be possible.

At this point it has to be mentioned that the CT and the 3D RA images should contain enough landmarks to allow for a reliable dataset registration within step S116. Thereby, the patient is supposed to lie fixed with regard to a table in order to further allow for a geometry-based registration between the 2D-X-ray dataset and the 3D RA dataset.

In this context the word “geometry” is used in the term “geometry-based registration” in order to denote the mechanical parts of a C-arm X-ray machine. Since a 3D RA dataset is produced by means of this machine respectively by a corresponding computer, the position of the data with regard to the machine is always known. Even if one moves the mechanical parts of the machine around the patient over many degrees of freedom, the positions of the parts of the machine are always known. When a 2D X-ray image is obtained with the same C-arm X-ray machine, based on the position of the mechanical parts of this machine, it is known how to project this 2D X-ray image on the 3D RA dataset. Therefore, the only constraint with geometry-based registration is that the patient does not move.

Further, it has to be mentioned that instead of a geometry-based registration between the 2D X-ray image and the 3D RA volume also an image-based registration would be possible. Though such an image-based registration tends to be more time-consuming and less robust, the image-based registration has the advantage that it relieves the patient under examination to be fixated during carrying out the steps S110 and S120, respectively.

Furthermore, it has to be mentioned also a hybrid registration approach would be possible. Thereby, a “geometrical” registration is used as a starting point for an image based registration. Such a hybrid registration can be used to correct for small movements, and is more robust than pure image based registration.

Following the above-mentioned data processing steps S116, S115, S126 and S125 there are carried out three further data processing steps S130, S140a and S140b.

First, as indicated with step S130, the position of the catheter tip is identified within a 3D representation of the patient's vessel structure. Thereby, information regarding the tracked catheter tip (see S125), information being derived from the registering step S126 and the a-priori knowledge that the catheter always is located within the vessel tree, which was segmented in the 3D RA dataset (see S115) are combined.

Second, as indicated with step S140a, a perpendicular view to the tracked catheter tip is generated. Thereby, the knowledge of the catheter tip position in 3D (see step S130) and the segmented vessel tree of the 3D RA representation (see S115) are combined.

Third, as indicated with step S140b, an improved perpendicular view to the tracked catheter tip is generated. In addition to the perpendicular view obtained with step S140a, which shows predominantly a cross sectional view of the corresponding vessel at the catheter tip position, the improved perpendicular view is extended to the soft tissue surrounding the vessel. In order generate an image showing precisely both the interior of the vessel and the tissue surrounding the vessel, the dataset representing the perpendicular view obtained with step S140a is combined with a dataset obtained within the registering step S116.

FIG. 2 shows a temporal workflow for carrying out the preferred embodiment of the invention. The workflow starts with a step S200, which is the step S100 illustrated in FIG. 1. The workflow ends with a step S240, which represents both the step S140a and the step S140b, which are both illustrated in FIG. 1. Also the intermediate steps S210, S215, S216, S220, S225, S226 and S230 are the same as the corresponding steps illustrated in FIG. 1. Therefore, the procedure for obtaining a perpendicular view to the catheter tip, wherein diagnostic scanning (CT), 3D RA and real time 2D X-ray imaging is combined, will not be explained in detail once more on the basis of the corresponding workflow.

The described method for generating a perpendicular view to the catheter tip, wherein CT, 3D RA and real time 2D X-ray imaging is combined, provides several advantages compared to state of the art procedures. In the following some of these advantages will be described briefly.

A) Known X-ray angiographic imaging provides only 2D and 3D information of the outer boundary of human residual lumen, which is in particular the outer boundary of iodinated contrast injected into the patient's vessel structure. Soft tissue information is not included. By contrast thereto, the described method allows for a precise understanding of 3D vessel anatomy with the highest possible contrast resolution along with the visualization of the characteristics of soft tissue surrounding the vessel structure.

B) The described method allows for precisely determining the position of the catheter tip with respect to the lesion position. Thereby, the position of the catheter tip may be acquired with interactive X-ray angiography. The position of the lesion is obtained either by CT, by magnetic resonance or by an X-ray soft tissue data scan.

C) The described method further allows for a visualization of a thrombus location with respect to the catheter position in endovascular thrombolytic therapy.

D) During a minimal-invasive interventional treatment of vascular pathologies and endovascular treatment of neoplastic tissue it is of great clinical benefit to obtain morphologic assessment of the tissue inside and surrounding the vessel, e.g. plaque, at the catheter tip position.

E) A further advantage of the described method is the fact that the catheter tip may be recognized in the 2D X-ray image. Thereafter, the catheter tip is projected on the 3D model of the vessels, which were segmented out of the 3DRA dataset. In this way one can obtain the 3D position and orientation of the catheter tip without moving the X-ray equipment. This means that a cross-section through the catheter tip position, with a normal corresponding to the tangent of the catheter tip, can be displayed real-time. Therefore, when a clinician moves the catheter, the cross-section moves along with it. Thereby, the tissue surrounding the catheter tip can be assessed precisely in real-time. The method can be accomplished without forcing the clinician to change his workflow and perform complex and time-consuming additional actions.

FIGS. 3a, 3b, and 3c show images, which are generated in the course of performing the preferred embodiment of the invention. Thereby, FIG. 3a shows an image depicting a 2D X-ray dataset registered with a 3D RA dataset. FIG. 3b shows an image depicting segmented vessels of a 3D RA dataset spatially registered with a corresponding CT dataset. FIG. 3c shows an image depicting a cross-sectional view of segmented vessels obtained by registering a 3D RA dataset with a CT dataset

FIG. 4 depicts an exemplary embodiment of a data processing device 425 according to the present invention for executing an exemplary embodiment of a method in accordance with the present invention. The data processing device 425 comprises a central processing unit (CPU) or image processor 461. The image processor 461 is connected to a memory 462 for temporally storing acquired or processed datasets. Via a bus system 465 the image processor 461 is connected to a plurality of input/output network or diagnosis devices, such as a CT scanner and a C-arm being used for 3D RA and for 2D X-ray imaging. Furthermore, the image processor 461 is connected to a display device 463, for example a computer monitor, for displaying images representing a perpendicular view to the inserted catheter reconstructed and registered by the image processor 461. An operator or user may interact with the image processor 461 via a keyboard 464 and/or any other output devices, which are not depicted in FIG. 4.

It should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

In order to recapitulate the above described embodiments of the present invention one can state:

It is described a method for determining the tissue surrounding an object being inserted into a patient. The method comprises acquiring a first dataset representing a first 3D image of the patient, acquiring a second dataset representing a second 3D image of the blood vessel structure of the patient and acquiring a third dataset representing a 2D image of the patient including the object. The method further comprises recognizing the object within the 2D image, registering two of the three datasets with each other in order to generate a first combined dataset, and registering the first combined dataset with the remaining dataset in order to generate a second combined dataset representing a further image surrounding the object. The method allows for combining diagnostic scanning such as CT, 3D RA and real-time 2D fluoroscopy. Thereby, it is possible to generate an image perpendicular to a catheter tip representing the object being inserted into the patient. Since the 3D-RA displays the lumen and the diagnostic scanning displays soft-tissue, it is possible to assess the tissue at the catheter tip position e.g. to identify soft plaque.

### LIST OF REFERENCE SIGNS

S100 obtain CT
S110 obtain 3D RA
S115 segment 3D RA
S116 register CT and 3D RA
S120 obtain 2D X-ray
S125 track catheter tip
S126 register 3D RA and 2D X-ray
S130 determine catheter tip position in 3D
S140a generate perpendicular view
S140b generate perpendicular view with CT
S200 obtain CT
S210 obtain 3D RA
S215 segment 3D RA
S216 register CT and 3D RA
S220 obtain 2D X-ray
S225 track catheter tip
S226 register 3D RA and 2D X-ray
S230 determine catheter tip position in 3D
S240 generate perpendicular view
326 image based on 2D X-ray dataset registered with 3D RA dataset
316 image of segmented vessels based on 3D RA dataset registered with CT dataset
340 image depicting cross sectional view of vessels based on 3D RA dataset registered with CT dataset
460 data processing device
461 central processing unit/image processor
462 memory
463 display device
464 keyboard
465 bus system

1. A method for determining and assessing the tissue surrounding an object being inserted into a patient, the method comprising the steps of
acquiring a first dataset representing a first three-dimensional image of the patient,
acquiring a second dataset representing a second three-dimensional image of the blood vessel structure of the patient,
acquiring a third dataset representing a two-dimensional image of the patient including the object being inserted into the patient,
recognizing the object within the two-dimensional image,
registering two of the three datasets with each other in order to generate a first combined dataset, and
registering the first combined dataset with the remaining dataset in order to generate a second combined dataset representing a further image surrounding the object.

2. The method according to claim 1, wherein
the step of registering two of the three datasets with each other comprises
registering the third dataset with the second dataset in order to generate the first combined dataset representing an image surrounding the object, whereby the object is back-projected in the blood vessel structure, and wherein
the step of registering the first combined dataset with the remaining dataset comprises
registering the first combined dataset with the first dataset.

3. The method according to claim 1, wherein
the step of registering two of the three datasets with each other comprises
registering the first dataset with the second dataset in order to generate the first combined dataset, and wherein
the step of registering the first combined dataset with the remaining dataset comprises
registering the first combined dataset with the third dataset.

4. The method according to claim 1, wherein
the object is a catheter being inserted into a vessel of the patient.

5. The method according to claim 4, further comprising the step of
creating a cross-sectional view surrounding the catheter based on the second combined dataset.

6. The method according to claim 5, wherein
the cross-sectional view is oriented perpendicular to the tangent of a section of the vessel, in which section the catheter is inserted.

7. The method according to claim 1, wherein
the first dataset is obtained by means of computed tomography and/or by means of magnetic resonance.

8. The method according to claim 1, wherein
the first dataset is acquired before the object is inserted into the patient.

9. The method according to claim 1, wherein
the second dataset is obtained by means of three-dimensional rotational angiography.

10. The method according to claim 1, wherein
the second dataset is obtained by means of computed tomography angiography and/or magnetic resonance angiography.

11. The method according to claim 1, wherein
the second dataset is limited to a region of interest surrounding the object.

12. The method according to claim 1, wherein
the second dataset comprises segmented images of the patient's blood vessel structure.

13. The method according to claim 1, wherein
the first combined dataset represents a three-dimensional image.

14. The method according to claim 1, wherein
the third dataset is acquired by means of X-radiation.

15. The method according to claim 1, wherein
the second dataset and the third dataset are acquired by means of the same medical examination apparatus.

16. The method according to claim 1, wherein
the object is moved within the patient's blood vessel structure and
third datasets are acquired for different positions of the object, wherein each third dataset represents a two-dimensional image of the patient including the object being inserted into the patient, and
for each position of the object there is carried out a data evaluation, which data evaluation comprises
recognizing the object within the two-dimensional image, and
registering the third dataset with the second dataset in order to generate a first combined dataset representing an image surrounding the object, whereby the object is back-projected into the blood vessel structure.

17. A data processing device
for determining and assessing the tissue surrounding an object being inserted into a patient,
the data processing device comprising
a data processor, which is adapted for performing the method as set forth in claim 1, and
a memory for storing the acquired first dataset, the acquired second dataset, the acquired third dataset and the registering first combined dataset.

18. A computer-readable medium on which there is stored a computer program
for determining and assessing the tissue surrounding an object being inserted into a patient,
the computer program, when being executed by a data processor, is adapted for performing the method as set forth in claim 1.

19. A program element
for determining and assessing the tissue surrounding an object being inserted into a patient,
the program element, when being executed by a data processor, is adapted for performing the method as set forth in claim 1.
